(19) United States
(12) Patent Application Publication
(10) Pub. No.: US 2009/0022786 A1
(43) Pub. Date: Jan. 22, 2009

(54) Oral pharmaceutical dosage form and manufacturing method thereof

(75) Inventors: Fang-Chen Lee, Taichung (TW); Bin-Ken Chen, Taichung (TW); Han-Chiang Kuo, Taichung (TW); Chi-Huang Chen, Taichung (TW)

Correspondence Address: HOWREY LLP-CA, C/O IP DOCKETING DEPARTMENT, 2941 FAIRVIEW PARK DRIVE, SUITE 200, FALLS CHURCH, VA, 22042-2924, US

(73) Assignee: YUNG SHIN PHARM. IND. CO., LTD.

(21) Appl. No.: 12/010,829

(22) Filed: Jan. 30, 2008

Related U.S. Application Data
(60) Provisional application No. 60/929,878, filed on Jul. 16, 2007.

(51) Int. Cl. A61K 9/28; A61K 9/14; A61K 31/195; A61K 9/48; A61K 31/122; A61K 31/216

(52) U.S. Cl. 424/451; 424/490; 514/567; 514/690; 514/529; 424/474

(57) ABSTRACT

The present invention provides an coated oral pharmaceutical dosage form comprising a composition of a non-steroidal anti-inflammatory drug (NSAID) as multilayered spherical granule combined with a composition of prostaglandin, and a film coating. The present invention also provides a method for manufacturing the dosage form, which comprising the steps of preparing the compositions of NSAID and prostaglandin separately, combing the compositions to form a pharmaceutical dosage form, and coating the dosage form with a film coating.

### FIELD OF THE INVENTION

The present invention relates to an oral pharmaceutical dosage form comprising a composition of a non-steroidal anti-inflammatory drug (NSAID) as spherical granule combined with a composition of prostaglandin, and an optional film coating without prostaglandin as the outermost part of the dosage form. The present invention also provides a method for manufacturing the dosage form, which comprises the steps of preparing the compositions of NSAID and prostaglandin separately, combing the compositions to form a pharmaceutical dosage form, and optionally coating the dosage form with a film coating containing no prostaglandin.

### BACKGROUND OF THE INVENTION

Non-steroidal anti-inflammatory drugs, usually abbreviated to NSAIDs, are drugs with analgesic, antipyretic, and anti-inflammatory effects—they reduce pain, fever and inflammation. Most NSAIDs act as non-selective inhibitors of the enzyme cyclooxygenase, inhibiting both the cyclooxygenase-1 (COX-1) and cyclooxygenase-2 (COX-2) isoenzyme. Cyclooxygenase catalyses the formation of prostaglandins and thromboxane from arachidonic acid (itself derived from the cellular phospholipid bilayer by phospholipase A₂). Prostaglandins act (among other things) as messenger molecules in the process of inflammation. NSAIDs are usually indicated for the treatment of acute or chronic conditions where pain and inflammation are present.

The widespread use of NSAIDs has meant that the adverse effects of these relatively safe drugs have become increasingly prevalent. The two main adverse drug reactions (ADRs), associated with NSAIDs relate to gastrointestinal (GI) effects and renal effects of the agents. NSAID, such as diclofenac sodium (with the chemical name “2-[(2,6-dichlorophenyl)amino] benzene acetic acid, monosodium salt”), causes a dual insult on the GIT—the acidic molecules directly irritate the gastric mucosa; and the inhibition of physiologica cyclo-oxygenase-1 (COX-1) reduces the levels of protective prostaglandins. These effects are dose-dependent, and in many cases severe enough to pose the risk of ulcer perforation, upper gastrointestinal bleeding, and death, limiting the use of NSAID therapy.

Prostaglandin, such as misoprosrol, has been used for prevention of gastroduodenal ulcers caused by NSAID (Drug Ther Perspect 15(5):10-12, 2000). Misoprosrol protects the stomach lining by increasing mucus and bicarbonate production and by enhancing blood flow to the stomach. It is approved only for the prevention of NSAID-induced ulcers.

U.S. Pat. No. 5,601,843 disclosed a pharmaceutical tablet composition including a core of an NSAID selected from diclofenac and piroxicam which core is surrounded by a mantle coating of a prostaglandin, misoprosrol. The prior tablet composition provides an ease of delivery of an NSAID for its therapeutic value such as the alleviation of inflammation in a system, which limits the undesirable side affects of ulcerogenesis associated with such NSAID therapy.

In a higher temperature condition, diclofenac sodium will occur epimerization and convert to 8-isomer. However, diclofenac sodium is much more stable when present as a dispersion system in hydroxypropyl methyl cellulose. Moreover, diclofenac sodium is accessible to be destroyed by humidity and light, accompanied with color deterioration. Therefore, one of embodiments of the present invention provides an coated oral pharmaceutical dosage form comprising a composition of a non-steroidal anti-inflammatory drug (NSAID) as multi-layered spherical granule combined with a composition of prostaglandin, and a film coating as outermost part of the dosage form, wherein the film coating contains no prostaglandin. In one embodiment, the NSAID spherical granule according to the invention, the NSAID-coated core may be coated with first protective layer, enteric coating, and second protective layer sequentially for protecting it from the destruction by humidity and light and easy delivery to small intestine. The composition of prostaglandin (misoprostol) may be present as particle for mixing and tableting with the NSAID spherical granule, or as suspension for coating on the NSAID spherical granule to form a coated particle for further filling into a capsule.

According to one of embodiments of the invention, NSAID (diclofenac sodium) can be protected by the multi-layered coating, and the destruction by humidity and light can be reduced. After the oral administration of present dosage form, the misoprostol formulation is dispersed and distributed onto gastric wall to form a protective layer, and the diclofenac sodium composition is delivered into small intestine to act its anti-inflammatory function.

### SUMMARY OF THE INVENTION

The present invention provides an oral pharmaceutical dosage form comprising a composition of non-steroidal anti-inflammatory drug (NSAID) as granule combined with a prostaglandin formulation, wherein the granule comprises at least an inner part and at least a NSAID drug part.

In certain embodiments, this invention relates to an coated oral pharmaceutical dosage form comprising a composition of a non-steroidal anti-inflammatory drug (NSAID) as multi-layered spherical granule combined with a composition of prostaglandin, and a film coating as outermost part of the dosage form, wherein the film coating contains no prostaglandin.

In one embodiment of the present invention, the active ingredient of the NSAID drug part is selected from the group consisting of diclofenac, ketoprofen, indomethacin, tiaprofenic acid, piroxicam, flubiprofen, tenoxicam, meloxicam and salts and mixture thereof. In another embodiment, the active ingredient of the NSAID drug part is diclofenac sodium.

In one embodiment of the present invention, the active ingredient of prostaglandin formulation is selected from the group consisting of prostaglandin E1, prostaglandin E1 analog and mixture thereof. In another embodiment, the active ingredient of the prostaglandin formulation is misoprostol.

In one embodiment of the invention, the NSAID drug part may further comprise at least an excipient, a binding agent and an emulsifier. In another embodiment of the invention, the prostaglandin formulation may further comprise at least an excipient, an emulsifier, and a disintegrating agent.

In another aspect, this invention provides to an oral pharmaceutical tablet comprising a composition of diclofenac sodium granule combined with a misoprostol formulation, wherein the dosage form further comprises at least a prostaglandin-free film coating, the prostaglandin-free film coating packages the diclofenac sodium granules and the misoprostol formulation. The diclofenac sodium granules further comprises at least a inner part, drug part, first protective layer, enteric coating, and second protective layer, wherein the drug part surrounds the inner part, the first protective layer the drug part, surrounds the film coating, the enteric coating surrounds the film coating, and second protective layer coats onto the enteric coating.

In a further aspect, the present invention provides a method of manufacturing the oral pharmaceutical dosage form, which comprises the steps of: (a) preparing a granule comprising at least a inner part and at least a NSAID drug part; (b) preparing a formulation comprising at least a prostaglandin as a form of particle or suspension; (c) combining the granule from step (a) and the prostaglandin formulation from step (b) in a way of mixing, tableting or particle coating; and (d) coating the tablet or particle from step (c) with a film coating containing no prostaglandin.

In one embodiment, the way of combination in step (c) is mixing the granule with the prostaglandin formulation to form a mixture for tableting. In another embodiment, the NSAID-containing granule is produced as a form of coated core tablet by tableting a mixture of the inner core and the NSAID drug part.

### BRIEF DESCRIPTION OF THE DRAWINGS

FIG. 1 shows a configuration of the multi-layered NSAID granule of one embodiment of the invention. The NSAID drug layer surrounds the inner core, and the NSAID-coated core is coated with protective layer, enteric coating, and second protective layer sequentially.

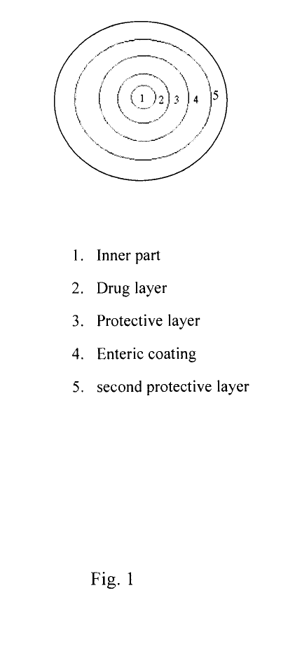

FIG. 2 shows the comparable plasma concentration profile of diclofenac sodium after oral administration of the coated tablet of one embodiment and reference drug, which is Pfizer's Arthrotec 75 Tablet.

FIG. 3 shows the plasma concentration profile of misoprostol after oral administration of the coated tablet of one embodiment and reference drug, which is Pfizer's Arthrotec 75 Tablet.

### DETAILED DESCRIPTION OF THE INVENTION

In one embodiment of the present invention, it provides an oral pharmaceutical dosage form comprising a composition of non-steroidal anti-inflammatory drug (NSAID), such as diclofenac sodium, for alleviating symptoms of inflammation, combined with a prostaglandin formulation for preventing gastric and duodenal ulcer induced by NSAID. In another embodiment of the present invention, the oral pharmaceutical dosage form comprises a composition of non-steroidal anti-inflammatory drug (NSAID) for alleviating symptoms of inflammation and at least an acid inhibitor formulation for preventing gastric and duodenal ulcer induced by NSAID. Aforementioned acid inhibitor may be selected from the group consisting of an H2 blocker such as cimetidine, ranitidine, ebrotidine, pabutidine, lafutidine, loxtidine and famotidine. Aforementioned acid inhibitor also can be selected from the proton pump inhibitor such as omeprazole, esomeprazole, lansoprazole, pantoprazole and rabeprazole.

Diclofenac sodium, which is a phenylacetic acid NSAID used in treating osteoarthritis and rheumatoid arthritis by the action of inhibiting cyclooxygenase, and therefore blocking the synthesis of prostaglandin from arachidonic acid, or by inhibiting lipoxygenase and further blocking the conversion of arachidonic acid to leukotrienes. Diclofenac sodium is soluble in methanol and ethanol and somewhat in water, but extremely insoluble in chloroform. Diclofenac sodium melts at 156˜158□ and is decomposed at the same time. Diclofenac sodium is accessible to be destroyed by humidity and light, accompanied with color deterioration.

Diclofenac sodium can be absorbed completely by gastrointestinal track, with about 50-60% entering systematic circulation. As orally administrating single dose of 50 mg, maximum plasma concentration was measured after 1-2 hours with peak value of 1.5 ug/ml. More than 90% of the active ingredient binds to plasma protein. Diclofenac sodium is mainly metabolized in liver and eliminated in glucuronate- or sulfate-associated form, ⅔ of which is eliminated with urine, and 1/30 with the faeces after biliary secretion. The half-life of iclofenac sodium is about 1-2 hours.

In the present embodiment, aforementioned prostaglandin can be misoprosrol. Misoprosrol, which has the chemical name of (±) methyl 11α,16-dihydroxy-16-methyl-9-oxoprost-13E-en-1-oateis a synthetic prostaglandin E1 derivative. The protective function of misoprosrol on gastric mucosa is to inhibit the secretion of gastric acid, stimulating the secretion of mucus, inhibit the secretion of HCO₃⁺, and improve the blood flow in mucosa. Misoprosrol is soluble in water and very unstable at room temperature. It is extremely sensitive to pH and temperature.

Misoprostol is rapidly absorbed after oral administration, maximum plasma concentration was measured after 15-30 minutes, inhibiting gastric acid secretion within 30 min. Misoprostol is de-esterified to misoprostol acid (the active metabolite) after absorbed in body. About 64-73% of misoprostol is eliminated with urine in 24 hours, and 15% with the faeces. The elimination half-life of misoprostol is about 20-40 minutes.

The present oral pharmaceutical dosage form comprises two major parts, a NSAID composition and a prostaglandin formulation. The NSAID composition may be formed as a granule with multi-layered configuration, which comprises:

(1) an inner part as the inmost part of the composition;

(2) a NSAID drug part surrounding the inner part;

(3) a first protective layer covering the N SAID drug part;

(4) an optional enteric coating surrounding the first protective layer; and

(5) an optional second protective layer, which coats onto the enteric coating.

The inner part used in the present invention may be made in a fluid bed centrifugal-type granulator (Glatt), or may be obtained from commercial producer as inner edible core. The inert core as the inner part of the dosage form is selected from the group consisting of sucrose, starch, talc, microcrystalline cellulose or a mixture thereof.

In one embodiment, the NSAID drug part may comprise at least one NSAID as active ingredient, excipient, binding agent, and emulsifier. In current invention, the active ingredient of the NSAID drug part may be at least one member selected from the group consisting of diclofenac, ketoprofen, indomethacin, tiaprofenic acid, piroxicam, flubiprofen, tenoxicam, meloxicam and salts thereof.

In another embodiment, the NSAID drug part may further comprise a disintegrating agent. The disintegrating agent may be a kind of saccharide, such as starch, lactose, mannose and the like. The NSAID drug part may further comprise a sliding agent. The exemplary sliding agents used in the invention are talc and colloidal silicon dioxide.

The emulsifier used in the invention may be selected from polyethylene (20) sorbitan monooleate, Tween 80, polyethylene ployprolene polymer, and sodium lauryl sulfate. The binding agent used in the invention may include polyvinylpyrrolidone, hydroxycellulose, hydroxypropyl methyl cellulose and the like.

The protective layer of the NSAID composition may be formed by hydroxypropyl methyl cellulose, hydroxypropyl cellulose, methyl cellulose or the mixture thereof. The protective layer may further comprise a light-covering agent, for example titanic oxide. The material forming the enteric coating may include methylacrylic acid-methyl methacrylate polymer, methylacrylic acid-ethyl acrylate polymer, hydroxymethyl cellulose phthalate and cellulose acetate.

The solvent used in preparing the NSAID composition may include alcohols, such as ethanol, propanol, and purified water.

In one embodiment, the NSAID composition may be prepared as follow:
1. polyvinylpyrrolidone and polyethylene(20) sorbitan monooleate are dissolved in purified water, and ethanol is added to the resulting solution;
2. preparation of the NSAID drug part: the mixture of sodium diclofenac (active ingredient), starch, talk powder and colloidal silicon dioxide is passed through 60-mesh sieve once and 40-mech sieve twice;
3. preparation of the first protective layer: hydroxypropyl methyl cellulose is dissolved in purified water with stirring, followed by the addition of polyethylene glycol and stirring to complete dissolution;
4. preparation of the enteric coating: methylacrylic acid-ethyl acrylate polymer is mixed with triethyl citrate and purified water;
5. preparation of the second protective layer: hydroxypropyl methyl cellulose is dissolved in purified water with stirring, followed by the addition of polyethylene glycol and stirring to complete dissolution, and titanic oxide which has been passed through 325-mech sieve once is added;
6. in a micro-granulating machine, the inner parts are placed and spray coated with the solution of binding agent prepared in step 1, and then the powder of NSAID drug part prepared in step 2 is added to bind onto the inner part. Repeat the operation of coating and binding to get a NSAID drug-containing granule.
7. in a fluid bed centrifugal-type granulator (Glatt), the NSAID drug-containing granules obtained in step 6 are placed and spray coated with the solution of protective layer prepared in step 3. After drying, a NSAID drug layer-containing granule coated by protective layer is obtained. Optionally, the granules are sprayed and coated with the mixture of enteric coating prepared in step 4, and dried to get granule with enteric coating on the outmost part in multi-layered configuration. Optionally, the multi-layered granules are sprayed and coated with the solution of second protective layer prepared in step 5. After drying, a multi-layered granule coated by a further protective layer is obtained.

In one embodiment, the prostaglandin formulation may be prepared as follow:
1. misoprostol (as 1% dispersed powder in hydroxymethyl cellulose) is dissolved in purified water with stirring, followed by the addition of polyethylene(20) sorbitan monooleate and mixing thoroughly;
2. the mixture of excipient comprising lactose, starch and microcrystalline cellulose is passed through 60-mesh sieve once and 40-mech sieve twice;
3. the mixture of sliding agent (colloidal silicon dioxide), lubricant (magnesium stearate) and disintegrating agent (cross linked povidone) is passed through 60-mesh sieve once and 40-mech sieve twice;
4. in a super-mixer machine, the mixture obtained in step 2 is placed, and the solution obtained in step 1 is added. After mixed thoroughly, the mixture is wet-milled with 16-mesh sieve once, and then dried at 50° C.; and
5. the milled granules obtained in step 4 are mixed with the mixture in step 3, and the mixture is milled with 25-mesh sieve three times.

In certain embodiments, provided are oral pharmaceutical dosage forms presented as tablets, with or without film coating. The tablet of the invention may be produced by mixing the multi-layered diclofenac sodium granules and misoprostol particles as prepared above, and milling the mixture with 18-mesh sieve three times before tableting. The obtained tablet each has weight of 730 mg, diameter of 12.1 m/m, hardness of 6˜12 kg/cm², thickness of 5.6˜6.0 m/m, disintegrating time <5 min, and brittleness <0.8%.

In another embodiment, the present invention provides a coated tablet comprising diclofenac sodium granules, misoprostol particles and a film coating as outermost part of the tablet. The film coating typically contains no prostaglandin. The film coating may comprise film-forming agent, such as hydroxypropyl methyl cellulose and hydroxypropyl cellulose; plasticizer, such as polyethylene glycol, triethyl citrate and glycerin triacetate; and light-covering agent such as titanic oxide.

The coated tablet may be produced as follow:
1. preparation of film coating: hydroxypropyl methyl cellulose is dissolved in purified water, then polyethylene glycol is added and stirred to complete dissolution, titanic oxide which has been passed through 325-mesh sieve once is added to the resulting solution, and followed by the addition of isopropanol to form a homogenous suspension;
2. in a film coating machine, the tablets described above are placed and spray coated with the suspension prepared in step 1. After drying, coated tablets comprising diclofenac granules and misoprostol particles with film coating are obtained.

The coated tablets according to the present invention may be produced in any suitable granulating machine, such as, for example, fluid bed centrifugal-type granulator (Glatt), fluid bed spray granulator (Huttlin) and the like. Moreover, the coated tablets according to the present invention exhibit a dissolution rate profile agreeing the rules listed in American pharmacopoeia, 29th edition.

In certain embodiments, the oral pharmaceutical dosage form according to the invention may be encapsulated in a hard gelatin capsule, in which the NSAID composition and prostaglandin formulation are mixed with an inert solid diluent, for example, calcium carbonate, calcium phosphate or kaolin. In another embodiment, the capsules are filled with the oral pharmaceutical dosage form presented as a coated particle composed of the composition of NSAID, the prostaglandin formulation and a film coating, wherein the composition of NSAID is coated with the prostaglandin formulation, the film coating substantially exists as outmost part of coated particle.

In certain embodiments, the oral pharmaceutical dosage form according to the invention may be presented as a double-layered tablet. The double-layered tablet is consisted of NSAID core tablet, prostaglandin layer and a film coating. In the preparation of the double-layered tablet, the NSAID granule is produced as a form of coated core tablet by tableting a mixture of the inner part and the NSAID drug part, and coating the coated core tablet with the first protective layer, enteric coating and second protective layers sequentially.

The present oral pharmaceutical dosage form is useful in alleviating the stomach mucosa damage caused by NSAIDs. By the protection of multi-layered coating, the destruction of diclofenac sodium by humidity and light can be reduced. After the oral administration of present dosage form, the misoprostol formulation is firstly dispersed and distributed onto gastric wall to form a protective layer, and the diclofenac sodium composition is delivered into small intestine to act its anti-inflammatory function.

### EXAMPLES

The invention is further defined by reference to the following exemplary examples describing in detail the preparation of the dosage forms of the invention. The following exemplary examples are set forth to assist in understanding the invention and should not be construed as specifically limiting the invention described and claimed herein.

### Example 1

### Preparation of Coated Tablet

A coated tablet composition was prepared comparing NSAID granules combined with prostaglandin formulation (particle) and coated by a film coating component.

The coated tablet had the following composition.

| Formulation | Weight (g) or volume (ml) | |
|---|---|---|
| NSAID granule | | |
| Inner part | 522.5 | g |
| NSAID drug part: | | |
| Diclofenac sodium | 765 | g |
| Corn starch | 300 | g |
| Sodium starch glycolate | 150 | g |
| Colloidal silicon dioxide | 62.5 | g |
| Providone (PVP K-30) | 57.5 | g |
| Polyethylene 20 sorbitan monooleate | 12.5 | g |
| (Tween 80) | | |
| Ethanol (Alcohol) | 460 | ml |
| Purified water | 690 | g |
| Protective layer: | | |
| Hydroxypropyl methyl cellulose (H.P.M.C) | 50 | g |
| Polyethylene glycol (P.E.G.6000) | 5 | g |
| Purified water | 710 | g |
| Enteric coating: | | |
| methylacrylic acid/methyl methacrylate | 400 | g |
| polymer (Eudragit L100) | | |
| Triethyl citrate | 80 | g |
| Talc powder | 40 | g |
| Ethanol (Alcohol) | 3600 | ml |
| Purified water | 400 | g |
| Second protective layer: | | |
| Hydroxypropyl methyl cellulose (H.P.M.C) | 50 | g |
| Polyethylene glycol (P.E.G.6000) | 5 | g |
| Purified water | 710 | g |
| Prostaglandin formulation (particle) | | |
| Misoprostol (H.P.M.C 1% Dispersion) | 52.5 | g |
| Lactose | 73 | g |
| Corn starch | 343.875 | g |
| Microcrystalline cellulose (Avicel 101) | 542.025 | g |
| Colloidal silicon dioxide | 41.975 | g |
| Magnesium stearate | 9.125 | g |
| Crospovidone | 109.5 | g |
| Tween 80 | 28 | g |
| Purified water | 477 | g |
| Film coating: | | |
| Hydroxypropyl methyl cellulose (H.P.M.C) | 55 | g |
| Polyethylene glycol (P.E.G.6000) | 5.4 | g |
| Titanium Dioxide | 14.6 | g |
| Purified water | 165 | g |
| Isopropyl Alcohol | 935 | ml |

### Preparation Protocol:

A. Preparation of NSAID Granule

1. NSAID Drug Part:
(1) Polyvinylpyrrolidone and polyoxyethylene 20 sorbitan monooleate were dissolved in purified water, and ethanol was added to the resulting solution and mixed thoroughly;
(2) The mixture of sodium diclofenac (NSAID, active ingredient), corn starch, Sodium starch glycolate and colloidal silicon dioxide was passed through 60-mesh sieve once and 40-mech sieve twice;
(3) In a micro-granulating machine, inner parts were placed and spray coated with the solution of binding agent prepared in step 1, and then the powder of mixture prepared in step 2 was added to bind onto the inner part. After the coating and drying (50° C.), NSAID drug layer-containing granules were obtained.

2. First Protective Layer:
(1) Hydroxypropyl methyl cellulose was dissolved in purified water with stirring, followed by the addition of polyethylene glycol and stirring to complete dissolution;
(2) In a fluid bed centrifugal-type granulator (Glatt), the NSAID drug layer-containing granules were placed and spray coated with the solution of protective layer prepared in the previous step (1). After the coating and drying, NSAID drug layer-containing granules coated with protective layer were obtained.

3. Enteric Coating:
(1) Ethanol is mixed with purified water, and triethyl citrate was added to the alcohol/water mixture Methylacrylic acid-ethyl acrylate polymer was then added and dissolved with stirring; and talc was added and mixed thoroughly;
(2) In a fluid bed centrifugal-type granulator (Glatt), the first protective layer-containing granules were placed and spray coated with the suspension of enteric coating prepared in the previous step (1). After the coating and drying, multi-layered granules with enteric coating on the outmost part were obtained.

4. Second Protective Layer:
Optionally, the multi-layered granules are spray coated with the solution of second protective layer similarly prepared as first protective layer. After drying, a multi-layered granule coated by a further protective layer is obtained.

B. Preparation of Prostaglandin Formulation (Particle)
1. Misoprostol (as 1% dispersion in hydroxymethyl cellulose) was dissolved in purified water with stirring, followed by the addition of polyoxyethylene 20 sorbitan monooleate and mixing thoroughly;
2. The mixture of lactose, corn starch and microcrystalline cellulose was passed through 60-mesh sieve once and 40-mech sieve twice;
3. The mixture of colloidal silicon dioxide, magnesium stearate and Crospovidone (cross linked povidone) was passed through 60-mesh sieve once and 40-mech sieve twice;
4. In a super-mixer machine (SY-RM-120), the mixture obtained in step 2 was placed, and the solution obtained in step 1 is added. After mixed thoroughly, the mixture was wet-milled (Quadro, 1945) with 16-mesh sieve once, and then dried at 50° C.;
5. The milled granules obtained in step 4 were mixed with the mixture in step 3, and the resulting mixture was milled with 25-mesh sieve three times.

C. Preparation of Mixed Tablet of Diclofenac and Misoprostol

Protected sodium diclofenac-containing granule (625 g) was mixed with misoprostol-containing particle (1200 g). The resulting mixture was milled with 18-mesh sieve three times, and then tableted in a tablet press. The obtained tablet each has the characteristics as follows:

Its weight is about 730 mg; diameter is 12.1 m/m; hardness is from 6 to 12 kg/cm²; thickness is from 5.6 to 6.0 m/m; disintegrating time is within 5 min; and brittleness is lower than 0.8%.

D. Preparation of the Final Film Coated Tablet
1. Hydroxypropyl methyl cellulose was dissolved in purified water with stirring, and then polyethylene glycol was added and stirred to complete dissolution. Titanic oxide which had been passed through 325-mesh sieve once was added to the resulting solution, and followed by the addition of isopropanol to form a homogenous suspension;
2. In a film coating machine, the tablets resulting from step C (1825 g) were placed and spray coated with the suspension prepared in step 1. After drying, film coated tablets comprising diclofenac granules and misoprostol particles were obtained.

### Example 2

### Preparation of Coated Tablet

A coated tablet composition was prepared comparing NSAID granules combined with prostaglandin formulation (particle) and coated by a film coating component.

The coated tablet had the following composition.

| | Weight (g) or | |
|---|---|---|
| Formulation | volume (ml) | |
| NSAID granule | | |
| Inner part: | 573 | g |
| NSAID drug part (Its configuration is layer): | | |
| Diclofenac sodium | 918 | g |
| Corn starch | 60 | g |
| Sodium starch glycolate | 60 | g |
| Colloidal silicon dioxide | 75 | g |
| Providone (PVP K-30) | 60 | g |
| Tween 80 | 6 | g |
| Ethanol (Alcohol) | 480 | ml |
| Purified water | 720 | g |
| First protective layer: | | |
| Hydroxypropyl methyl cellulose (H.P.M.C) | 60 | g |
| Polyethylene glycol (P.E.G.6000) | 6 | g |
| Purified water | 852 | g |
| Enteric coating: | | |
| Methylacrylic acid/ethyl acrylate polymer | 3100 | g |
| (Spraypol L30D-55) | | |
| Triethyl citrate | 186 | g |
| Purified water | 775 | g |
| Second protective layer: | | |
| Hydroxypropyl methyl cellulose (H.P.M.C) | 60 | g |
| Polyethylene glycol (P.E.G.6000) | 6 | g |
| Purified water | 852 | g |
| Prostaglandin formulation (particle): | | |
| Misoprostol (H.P.M.C 1% Dispersion) | 52.5 | g |
| Lactose | 73 | g |
| Corn starch | 338.4 | g |
| Microcrystalline cellulose (Avicel 101) | 547.5 | g |
| Colloidal silicon dioxide | 41.975 | g |
| Magnesium stearate | 9.125 | g |
| Magnesium stearate | 9.125 | g |
| Crospovidone | 109.5 | g |
| Tween 80 | 28 | g |
| Purified water | 500 | g |
| Film coating for tablets resulting from step C (1825 g): | | |
| Hydroxypropyl methyl cellulose (H.P.M.C) | 55 | g |
| Polyethylene glycol (P.E.G.6000) | 5.4 | g |
| Titanium Dioxide | 14.6 | g |
| Purified water | 165 | g |
| Isopropyl Alcohol | 1210 | ml |

### Preparation Protocol:

A. Preparation of NSAID Granule:

The NSAID drug layer, protective layers and protected sodium diclofenac-containing granule were prepared as described in Example 1. The suspension of enteric coating was prepared by mixing methylacrylic acid/ethyl acrylate polymer, which had been passed through 100-mesh sieve once, and was mixed with triethyl citrate and purified water completely.

B. Preparation of Prostaglandin formulation (particle) The prostaglandin particle was prepared as described in Example 1.

C. Preparation of Mixed tablet

Protected sodium diclofenac-containing granule (625 g) was mixed with misoprostol-containing particle (1200 g). The resulting mixture was milled with 18-mesh sieve three times, and then tableted in a tablet press.

D. Preparation of Final Film Coated Tablet

The film coated tablets comprising diclofenac granules and misoprostol particles were prepared as described in Example 1.

### Example 3

### Preparation of Coated Tablet

A coated tablet composition was prepared comparing NSAID granules combined with prostaglandin formulation (particle) and coated by a film coating component.

The coated tablet had the following composition.

| | Weight (g) or | |
|---|---|---|
| Formulation | volume (ml) | |
| NSAID granule | | |
| Inner part | 592.5 | g |
| NSAID drug part: | | |
| Diclofenac sodium | 765 | g |
| Corn starch | 50 | g |
| Sodium starch glycolate | 50 | g |
| Colloidal silicon dioxide | 62.5 | g |
| Providone (PVP K-30) | 50 | g |
| Tween 80 | 5 | g |
| Ethanol (Alcohol) | 600 | ml |
| Purified water | 400 | g |
| First protective layer: | | |
| Hydroxypropyl methyl cellulose (H.P.M.C) | 75 | g |
| Polyethylene glycol (P.E.G.6000) | 7.5 | g |
| Purified water | 1070 | g |
| Enteric coating: | | |
| Spraypol L30D-55 | 2083.3 | g |
| Triethyl citrate | 125 | g |
| Purified water | 417 | g |
| Second protective layer: | | |
| Hydroxypropyl methyl cellulose (H.P.M.C) | 75 | g |
| Polyethylene glycol (P.E.G.6000) | 7.5 | g |
| Titanium dioxide | 10 | g |
| Purified water | 1070 | g |
| Prostaglandin formulation (particle): | | |
| Misoprostol (H.P.M.C 1% Dispersion) | 53.5 | g |
| Lactose | 109.5 | g |
| Corn starch | 328.9 | g |
| Microcrystalline cellulose (Avicel 101) | 547.5 | g |
| Colloidal silicon dioxide | 41.975 | g |
| Magnesium stearate | 9.125 | g |
| Crospovidone | 91.25 | g |
| Tween 80 | 18.25 | g |
| Purified water | 535 | g |
| Film coating for tablets resulting from step C (1825 g): | | |
| Hydroxypropyl methyl cellulose (H.P.M.C) | 55 | g |
| Polyethylene glycol (P.E.G.6000) | 5.4 | g |
| Titanium Dioxide | 14.6 | g |
| Purified water | 165 | g |
| Isopropyl Alcohol | 1210 | ml |

Preparation Protocol:

A. Preparation of NSAID Granule

The drug layer, protective layer and protected sodium diclofenac-containing granule were prepared as described in Example 1. The suspension of enteric coating was prepared as described in Example 2. The suspension of second protective layer was prepared as follow: hydroxypropyl methyl cellulose was dissolved in purified water with stirring, then polyethylene glycol was added and stirred to complete dissolution, and titanic oxide which had been passed through 325-mesh sieve once was added to form a suspension.

B. Preparation of Prostaglandin Formulation (Particle)

The prostaglandin particle was prepared as described in Example 1.

C. Preparation of Mixed Table

Protected sodium diclofenac-containing granule (625 g) was mixed with misoprostol-containing particle (1200 g). The resulting mixture was milled with 18-mesh sieve three times, and then tableted in a tablet press.

D. Preparation of Film Coated Tablet

The film coated tablets comprising diclofenac granules and misoprostol particles were prepared as described in Example 1.

### Example 4

### Preparation of Capsule

A capsule composition was prepared comparing NSAID granules coated with prostaglandin formulation (as outer membrane) filled into a capsule.

The coated tablet had the following composition.

| Formulation | Weight (g) or volume (ml) | |
|---|---|---|
| NSAID granule | | |
| Inner part | 660.5 | g |
| NSAID drug part: | | |
| Diclofenac sodium | 765 | g |
| Corn starch | 60 | g |
| Sodium starch glycolate | 60 | g |
| Colloidal silicon dioxide | 63 | g |
| Providone (PVP K-30) | 49.5 | g |
| Tween 80 | 6 | g |
| Ethanol (Alcohol) | 549 | ml |
| Purified water | 396 | g |
| First protective layer: | | |
| Hydroxypropyl methyl cellulose (H.P.M.C) | 75 | g |
| Polyethylene glycol (P.E.G.6000) | 7.5 | g |
| Purified water | 1070 | g |
| Enteric coating: | | |
| Spraypol L30D-55 | 2000 | g |
| Triethyl citrate | 120 | g |
| Purified water | 400 | g |
| Second protective layer: | | |
| Hydroxypropyl methyl cellulose (H.P.M.C) | 75 | g |
| Polyethylene glycol (P.E.G.6000) | 7.5 | g |
| Titanium dioxide | 9 | g |
| Purified water | 1070 | g |
| Prostaglandin formulation (suspension): | | |
| Misoprostol (H.P.M.C 1% Dispersion) | 214 | g |
| Corn starch | 90 | g |
| Tween 80 | 105 | g |
| Titanium dioxide | 15 | g |
| Purified water | 2520 | g |
| Talc | 18 | g |

### Preparation Protocol:

A. Preparation of NSAID Granule

The drug layer, protective layer and protected sodium diclofenac-containing granule were prepared as described in Example 1. The suspension of enteric coating was prepared as described in Example 2. The suspension of second protective layer was prepared as described in Example 3.

B. Preparation of Prostaglandin Formulation (Suspension)

Misoprostol (as 1% dispersion in hydroxymethyl cellulose) was dissolved in purified water with stirring, followed by the addition of polyoxyethylene 20 sorbitan monooleate and mixing thoroughly. Corn starch was added and mixed thoroughly, and then titanic oxide which had been passed through 325-mesh sieve once was added to form a suspension;

In a fluid bed centrifugal-type granulator (Glatt), the protected sodium diclofenac-containing granules were placed and spray coated with the suspension of misoprostol prepared in the step B. After drying, a sodium diclofenac-containing granule coated with misoprostol drug layer was obtained. Talc was added to the granules and mixed thoroughly.

C. Capsule Filling

The sodium diclofenac-containing granules coated with misoprostol drug layer obtained by step B. were encapsulated in No. 2 capsules at the amount of 300 mg/capsule. (BOSH., GKF-400)

### Example 5

### Preparation of Coated Core Tablet

A coated core tablet composition was prepared comparing NSAID core combined with prostaglandin formulation (particle) and coated by a film coating component.

The coated core tablet had the following composition.

| Formulation | Weight (g) or volume (ml) | |
|---|---|---|
| NSAID granule | | |
| NSAID drug part: | | |
| Diclofenac sodium | 765 | g |
| Corn starch | 375 | g |
| Microcrystalline cellulose (Avicel 101) | 243 | g |
| Colloidal silicon dioxide | 15 | g |
| Magnesium stearate | 15 | g |
| Providone (PVP K-30) | 12 | g |
| Crospovidone | 75 | g |
| Purified water | 300 | g |
| First protective layer: | | |
| Hydroxypropyl methyl cellulose (H.P.M.C) | 60 | g |
| Polyethylene glycol (P.E.G.6000) | 6 | g |
| Titanium dioxide | 4.5 | g |
| Purified water | 750 | g |
| Enteric coating: | | |
| Spraypol L30D-55 | 425 | g |
| Triethyl citrate | 25.5 | g |
| Purified water | 85 | g |
| Second protective layer: | | |
| Hydroxypropyl methyl cellulose (H.P.M.C) | 52.5 | g |
| Polyethylene glycol (P.E.G.6000) | 4 | g |
| Purified water | 660 | g |
| Prostaglandin formulation (particle): | | |
| Misoprostol (H.P.M.C 1% Dispersion) | 210 | g |
| Lactose | 730 | g |
| Corn starch | 1368 | g |
| Microcrystalline cellulose (Avicel 101) | 2555 | g |
| Colloidal silicon dioxide | 182.5 | g |
| Magnesium stearate | 36.5 | g |
| Crospovidone | 365 | g |
| Tween 80 | 73 | g |
| Purified water | 2330 | g |
| Film coating for the core tablets resulting | | |
| from step C (7300 g) | | |
| Hydroxypropyl methyl cellulose (H.P.M.C) | 110 | g |
| Polyethylene glycol (P.E.G.6000) | 10.8 | g |
| Titanium Dioxide | 29.2 | g |
| Purified water | 330 | g |
| Isopropyl Alcohol | 2420 | ml |

Preparation Protocol:

A. Preparation of NSAID coated tablet
1. Providone was dissolved in purified water with stirring;
2. A mixture of diclofenac sodium and corn starch was passed through 60-mesh sieve once and 40-mech sieve twice;
3. A mixture of magnesium stearate, colloidal silicon dioxide and crospovidone was passed through 60-mesh sieve once and 40-mech sieve twice;
4. In a super-mixer machine, the mixture obtained in step 2 was placed, and the solution obtained in step 1 is added. After mixed thoroughly, the mixture was wet-milled with 16-mesh sieve once, and then dried at 50° C.;
5. The milled granules obtained in step 4 were mixed with the mixture resulted from step 3 and microcrystalline cellulose, and the resulting mixture was milled with 25-mesh sieve three times;
6. The milled granules obtained in step 4 were tableted in a tablet press. The obtained tablet each has weight of 150 mg, diameter of 7.4 m/m, hardness of 5˜7 kg/cm², thickness of 3.5-3.7 m/m, disintegrating time <15 min, and brittleness <0.8%.
7. In a film coating machine, the diclofenac sodium-containing tablets obtained above were placed and spray coated with the suspension of protective layer. After drying, second protective layer-coated core tablets comprising diclofenac sodium were obtained.
8. In a film coating machine, second protective layer-coated tablets obtained in step 7 were placed and spray coated with the suspension of enteric coating. After drying, coated core tablets with enteric coating were obtained.
9. The coated tablets with enteric coating were coated with second protective layer to produce protected d iclofenac sodium-containing tablets.

B. Preparation of Prostaglandin Formulation (Particle)

The prostaglandin particle was prepared as described in Example 1.

C. Tableting

Protected sodium diclofenac-containing tablet (1780 g) and misoprostol containing particle (5520 g) were tableted in a multi-layer tablet press. The obtained coated core tablet each has weight of 730 mg, diameter of 12.1 m/m, hardness of 6˜12 kg/cm², thickness of 5.6˜6.0 m/m, disintegrating time <5 min, and brittleness <0.8%.

D. Preparation of Film Coated Tablet
In a film coating machine, the core tablets resulting from step C (7300 g) were placed and spray coated with the suspension prepared as described in example 1. After drying, film coated core tablets comprising diclofenac granules and misoprostol particles were obtained.

### Example 6

### Preparation of Coated Core Tablet

A coated core tablet composition was prepared comparing NSAID core combined with prostaglandin formulation (particle) and coated by a film coating component.

The coated core tablet had the following composition.

| Formulation | Weight (g) or volume (ml) | |
|---|---|---|
| NSAID granule | | |
| NSAID drug part: | | |
| Diclofenac sodium | 765 | g |
| Corn starch | 375 | g |
| Microcrystalline cellulose (Avicel 101) | 243 | g |
| Colloidal silicon dioxide | 15 | g |
| Magnesium stearate | 15 | g |
| Providone (PVP K-30) | 12 | g |
| Crospovidone | 75 | g |
| Purified water | 300 | g |
| First protective layer: | | |
| Hydroxypropyl methyl cellulose (H.P.M.C) | 60 | g |
| Polyethylene glycol (P.E.G.6000) | 6 | g |
| Titanium dioxide | 4.5 | g |
| Purified water | 750 | g |
| Enteric coating: | | |
| Spraypol L30D-55 | 375 | g |
| Triethyl citrate | 25.5 | g |
| Purified water | 75 | g |
| Second protective layer: | | |
| Hydroxypropyl methyl cellulose (H.P.M.C) | 52.5 | g |
| Polyethylene glycol (P.E.G.6000) | 4 | g |
| Purified water | 660 | g |
| Prostaglandin formulation (particle): | | |
| Misoprostol (H.P.M.C 1% Dispersion) | 210 | g |
| Lactose | 730 | g |
| Corn starch | 1388 | g |
| Microcrystalline cellulose (Avicel 101) | 2555 | g |
| Colloidal silicon dioxide | 182.5 | g |
| Magnesium stearate | 36.5 | g |
| Crospovidone | 365 | g |
| Tween 80 | 73 | g |
| Purified water | 2330 | g |
| Film coating for the core tablets resulting | | |
| from step C (7300 g) | | |
| Hydroxypropyl methyl cellulose (H.P.M.C) | 110 | g |
| Polyethylene glycol (P.E.G.6000) | 10.8 | g |
| Titanium Dioxide | 29.2 | g |
| Purified water | 330 | g |
| Isopropyl Alcohol | 2420 | ml |

Preparation Protocol:

A. Preparation of NSAID coated tablet
1. The diclofenac sodium-containing tablets were obtained in the way as described in Example 5. Each tablet has weight of 150 mg, diameter of 7.4 m/m, hardness of 5˜7 kg/cm², thickness of 3.5˜3.7 m/m, disintegrating time <15 min, and brittleness <0.8%;
2. The coating procedures with first protective layer, enteric coating and second protective layer were performed sequentially as described in Example 5, and a protected diclofenac sodium-containing tablet was obtained.

B. Preparation of Prostaglandin Formulation (Particle)

The prostaglandin particle was prepared as described in Example 1.

C. Tableting

Coated core tablets comprising the protected sodium diclofenac-containing tablet (1780 g) and misoprostol-containing particle (5520 g) were tableted and obtained as described in Example 5.

D. Preparation of Film Core Coated Tablet

The film coated core tablets comprising diclofenac granules and misoprostol particles were prepared as described in Example 5.

### Dissolution Rate Profile Testing:

The dissolution rate of diclofenac sodium and misoprostol in the pharmaceutical dosage form prepared in example 1 to 6 and a commercial product (Arthrotec 75 Tablet, Pfizer) were determined in accordance with the standard protocol described in American pharmacopoeia, 29th edition (Diclofenac sodium Delayed-release Tablets, P683. 2006). The comparable results were listed in Table 1 and 2, respectively. (Diclofenac sodium: Medium: 0.1N HCL 2 hrs→PH6.8 1 hrs, Apparatus 2 (Paddle): 100 rpm, Detector: 278 nm. Misoprostol: H2O 500 ml. Apparatus 2 (Paddle): 50 rpm. LC/MS/MS)

TABLE 1
The dissolution rate of diclofenac sodium

Time and pH

| | |
|---|---|
| 0.1N HCl (120 min) | pH 6.8 (45 min) |
| 100 rpm | 100 rpm |

| | |
|---|---|
| Example | Dissolution (%) |

| | | |
|---|---|---|
| 1 | 1.13% | 90.4% |
| 2 | 2.47% | 103.97% |
| 3 | 1.77% | 105.02% |
| 4 | 1.39% | 110.64% |
| 5 | 0.0% | 94.01% |
| 6 | 0.0% | 104.04% |
| Commercial product | 0.04% | 89.59% |

As the results listed in Table 1, the pharmaceutical dosage form produced by the method of the invention can provide good or satisfactory dissolution rate of diclofenac sodium, as compared with the commercial product.

TABLE 2
The dissolution rate of misoprostol

| | Time and solvent |
|---|---|
| | H₂O (30 min) 50 rpm |
| Example | Dissolution |
| 1 | 89.12% |
| 2 | 88.2% |
| 3 | 95.87% |
| 4 | 91.3% |
| 5 | 92.4% |
| 6 | 93.5% |
| Commercial product | 94.0% |

As the results listed in Table 2, the pharmaceutical dosage form produced by the method of the invention can provide good or satisfactory dissolution rate of misoprostol, as compared with the commercial product.

### Plasma Concentration Profile Testing

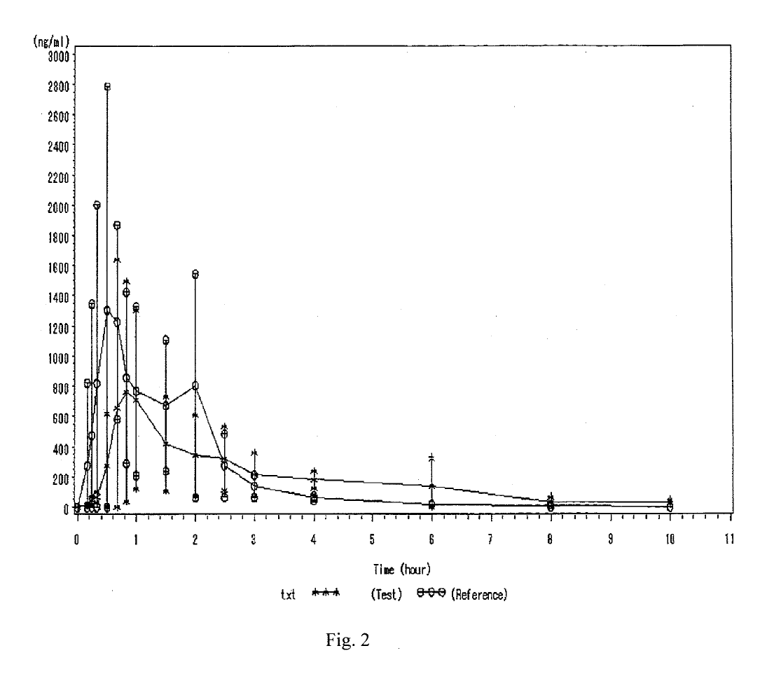

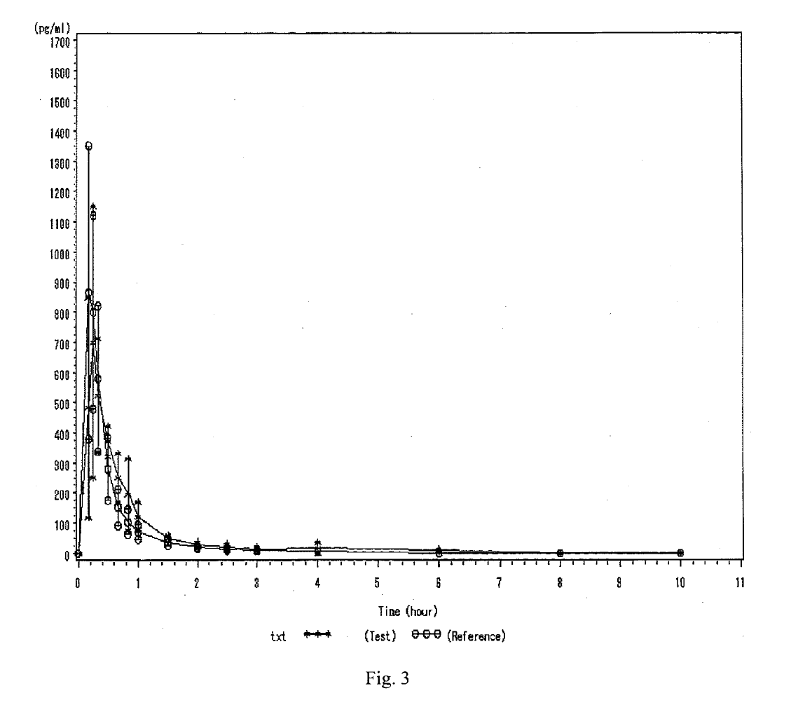

The drug absorption rate as presented in plasma concentration profiles of diclofenac sodium and misoprostol in the coated tablet in Example 1 were tested, and compared with those of a commercial tablet (Arthrotec 75 Tablet. Pfizer). The results are showed in FIG. 2 and FIG. 3. (Diclofenac sodium: Medium: 0.1 NHCL 2 hrs→PH 6.8 1 hrs. Apparatus 2 (Paddle): 100 rpm. Detector: 278. Misoprostol: H2O 500 ml. Apparatus 2 (Paddle): 50 rpm. LC/MS/MS)

As showed in FIG. 2, the coated tablet as prepared in Example 1 exhibited a satisfactory absorption rate of diclofenac sodium, as compared to the commercial product. The inventors achieve the bioequivalent test and get the good result of absorption rate by enabling the present invention. A similar result was showed in FIG. 3. The coated tablet as prepared in Example 1 exhibited an excellent absorption rate of misoprostol, as compared to the commercial product.

The foregoing description is considered as illustrative only of the principles of the invention. It will be apparent to those skilled in the art that many modifications, both to materials and methods, may be practiced without departing from the purpose and interest of this invention. Accordingly, all suitable modifications and equivalents may be considered to fall within the scope of the invention as defined by the claims that follow.

1. An oral pharmaceutical dosage form comprising a composition of non-steroidal anti-inflammatory drug (NSAID) as granule combined with a prostaglandin formulation, wherein the granule comprises at least an inner part and at least a NSAID drug part.

2. The oral pharmaceutical dosage form of claim 1, wherein the dosage form further comprises at least a film coating as outermost part of the dosage form, and the NSAID drug part surrounds the inner part.

3. The oral pharmaceutical dosage form of claim 2, wherein the film coating comprises no prostaglandin.

4. The oral pharmaceutical dosage form of claim 3, wherein the granule further comprises first protective layer, the first protective layer being as outermost part of the granule.

5. The oral pharmaceutical dosage form of claim 4, wherein the configuration of the granule is multilayer.

6. The oral pharmaceutical dosage form of claim 4, wherein the granule further comprises at least an enteric coating and second protective layer, wherein the enteric coating surrounds first protective layer and second protective layer coats onto the enteric coating.

7. The oral pharmaceutical dosage form of claim 1, wherein the active ingredient of the NSAID drug part is selected from the group consisting of diclofenac, ketoprofen, indomethacin, tiaprofenic acid, piroxicam, flubiprofen, tenoxicam, meloxicam and salts and mixture thereof.

8. The oral pharmaceutical dosage form of claim 1, wherein the active ingredient of the NSAID drug part is diclofenac sodium.

9. The oral pharmaceutical dosage form of claim 1, wherein the active ingredient of prostaglandin formulation is selected from the group consisting of prostaglandin E1, prostaglandin E1 analog and mixture thereof.

10. The oral pharmaceutical dosage form of claim 1, wherein the active ingredient of the prostaglandin formulation is misoprostol.

11. The oral pharmaceutical dosage form of claim 6, wherein the NSAID drug part further comprises at least an excipient, a binding agent and an emulsifier.

12. The oral pharmaceutical dosage form of claim 11, wherein the prostaglandin formulation further comprises at least an excipient, an emulsifier, and a disintegrating agent.

13. The oral pharmaceutical dosage form of claim 12, wherein enteric coating, the first and/or second protective layer further comprises at least a light-covering agent.

14. The oral pharmaceutical dosage form of claim 13, wherein the light-covering agent is titanic oxide.

15. The oral pharmaceutical dosage form of claim 1, which is presented as a coated tablet.

16. The oral pharmaceutical dosage form of claim 1, wherein the composition of NSAID and the prostaglandin formulation are mixed in a tablet.

17. The oral pharmaceutical dosage form of claim 1, wherein the composition of NSAID is dispersed in the prostaglandin formulation.

18. The oral pharmaceutical dosage form of claim 15, wherein the composition of NSAID is in a form of coated core tablet.

19. The oral pharmaceutical dosage form as claim 1, which is presented as a coated particle composed of the composition of NSAID and the prostaglandin formulation, wherein the composition of NSAID is coated with the prostaglandin formulation.

20. The oral pharmaceutical dosage form as claim 19, which the coated particles are further filled in a capsule.

21. An oral pharmaceutical tablet comprising a composition of diclofenac sodium granule combined with a misoprostol formulation, wherein the dosage form further comprises at least a prostaglandin-free film coating, the prostaglandin-free film coating package the diclofenac sodium granule and the misoprostol formulation, the diclofenac sodium granule further comprises at least a inner part, drug part, first protective layer and enteric coating, the drug part surrounds the inner part, the enteric coating surrounds the film coating and second protective layer coats onto the enteric coating.

22. A method for manufacturing the oral pharmaceutical dosage form of claim 1, comprising the steps of:
(a) preparing a granule comprising at least a inner part and at least a NSAID drug part;
(b) preparing a formulation comprising at least a prostaglandin as a form of particle or suspension;
(c) combining the granule from step (a) and the prostaglandin formulation from step (b) in a way of mixing, tableting or particle coating; and
(d) coating the tablet or particle from step (c) with a film coating containing no prostaglandin.

23. The method of claim 22, wherein the active ingredient of the NSAID drug part is selected from the group consisting of diclofenac, ketoprofen, indomethacin, tiaprofenic acid, piroxicam, flubiprofen, tenoxicam, meloxicam and salts and mixture thereof.

24. The method of claim 22, wherein the active ingredient of the NSAID drug part is diclofenac sodium.

25. The method of claim 22, wherein the active ingredient of prostaglandin formulation is selected from the group consisting of prostaglandin E1, prostaglandin E1 analog and mixture thereof.

26. The method of claim 22, wherein the active ingredient of the prostaglandin formulation is misoprostol.

27. The method of claim 22, wherein the granule is multilayered spherical granule.

28. The method of claim 22, wherein the way of combination in step (c) is mixing the granule with the prostaglandin formulation to form a mixture for tableting.

29. The method of claim 28, wherein the granule is produced as a form of coated core tablet by tableting a mixture of the inner part and the NSAID drug part, and coating the coated core tablet with the first protective layer, enteric coating and second protective layers sequentially.

30. An oral pharmaceutical dosage form comprising a composition of non-steroidal anti-inflammatory drug (NSAID) as granule combined with at least an acid inhibitor formulation, wherein the granule comprises at least an inner part and at least a NSAID drug part, the NSAID drug part surrounds the inner part, the dosage form further comprises at least a film coating containing no prostaglandin and being as outermost part of the dosage form

31. The oral pharmaceutical dosage form of claim 30, wherein said acid inhibitor is selected from the group consisting of an H2 blocker or a proton pump inhibitor.

32. The oral pharmaceutical dosage form of claim 31, wherein said H2 blocker is selected from the group consisting of cimetidine, ranitidine, ebrotidine, pabutidine, lafutidine, loxtidine and famotidine.

33. The oral pharmaceutical dosage form of claim 31, wherein said acid inhibitor is a proton pump inhibitor selected from the group consisting of omeprazole, esomeprazole, lansoprazole, pantoprazole and rabeprazole.
